(19) United States
(12) Patent Application Publication
(10) Pub. No.: US 2012/0303079 A1
(43) Pub. Date: Nov. 29, 2012

(54) APPARATUS AND METHOD FOR COMBINED CARDIAC FUNCTION MANAGEMENT AND RENAL THERAPIES

(76) Inventors: Deepa Mahajan, Circle Pines, MN (US); Ramesh Wariar, Blaine, MN (US); Shibaji Shome, Arden Hills, MN (US)

(21) Appl. No.: 13/477,148

(22) Filed: May 22, 2012

Related U.S. Application Data
(60) Provisional application No. 61/488,989, filed on May 23, 2011.

(51) Int. Cl. A61N 1/365

(52) U.S. Cl. 607/14; 607/17

(57) ABSTRACT

A system can coordinate operation of a cardiac function management (CFM) device and a renal device, such as during a vulnerable period in which a patient has an increased risk of tachyarrhythmia.

### CLAIM OF PRIORITY

This application claims the benefit of priority under 35 U.S.C. §119(e) of Mahajan et al., U.S. Provisional Patent Application Ser. No. 61/488,898, entitled “APPARATUS AND METHOD FOR COMBINED CARDIAC FUNCTION MANAGEMENT AND RENAL THERAPIES”, filed on May 23, 2011, which is herein incorporated by reference in its entirety.

### BACKGROUND

An implantable or other ambulatory medical device, such as a cardiac function management device (CFM), can be used to treat congestive heart failure (CHF or HF) or another heart disorder. Examples of CFM devices can include a pacemaker, an implantable cardioverter defibrillator (ICD), a cardiac resynchronization therapy (CRT) device, a cardiac contractility modulation (CCM) device, a cardiovascular function related neuromodulation device, or a combination device, such as providing a combination of one or more of the above.

A renal device can include an external electromechanical system, such as to provide renal therapy, such as renal replacement therapy. A renal device can include a blood pump that can circulate blood through a blood circuit, which can include a hemodialyzer filter. In an example, the renal device can be used to provide dialysis, which can include removing small solutes, such as across a semi permeable membrane from a side of higher concentration (e.g., blood side) to a side of lower concentration. In an example, the renal device can be used to provide ultrafiltration, which can include removal of water and small-to-medium solutes (e.g., by convection), such as through the application of a hydrostatic pressure across a semi-permeable membrane. In an example, the renal device can be used to provide hemofiltration, which can combine ultrafiltration with fluid replacement. The term “clearance” can be used to describe the complete removal of a substance from a specific volume of blood per unit time. In an example, the renal device can combine dialysis, ultrafiltration, hemofiltration, or one or more other renal therapies.

Wariar et al. U.S. Patent Publication No. 2007/0175827, entitled CARDIAC FUNCTION MANAGEMENT DEVICE AND SENSOR-SUITE FOR THE OPTIMAL CONTROL OF ULTRAFILTRATION AND RENAL REPLACEMENT THERAPIES, which is incorporated herein by reference, refers to a cardiorenal patient monitoring system with either implanted or non-implanted device(s), remote peripheral device(s), computer network(s), host, and communication means between the device(s), computer network(s), and host useful with an implanted cardiac device and a dialysis machine in renal therapy. (See Wariar et al. at Abstract.)

Gill et al. U.S. Pat. No. 7,529,580, entitled DETECTION OF RENAL FAILURE BY CARDIAC IMPLANTABLE MEDICAL DEVICE, refers to tracking morphological features within electrical cardiac signals and monitoring feature changes to detect renal failure. (See Gill et al. at Abstract.)

### OVERVIEW

The present inventors have recognized, among other things, that patients with chronic kidney disease (CKD) have more risk of arrhythmias and sudden cardiac death (SCD) than the general population. The present inventors have also recognized that the most common cause of death of such CKD patients is cardiac-related. Moreover, an increasing proportion of HF patients have CKD (e.g., estimated glomerular filtration rate (eGFR) less than 60 ml/min/m²) and 30% of such HF patients experience worsening renal function (e.g., D[Cr]>0.3 mg/dl) at a HF hospitalization. About 5% of patients with implanted CFM devices are on dialysis.

The present inventors have also recognized that patients on dialysis are at increased risk of cardiac arrhythmias during dialysis, and even following dialysis. This can be due to, for example, increased premature ventricular contractions (PVCs), increased autonomic tone and decreased heart rate variability (HRV), or changes in serum potassium at dialysis, but CFM devices are typically not aware of these periods of increased tachyarrhythmia vulnerability to modify monitoring or therapy accordingly. The present inventors have recognized that if the GEM device can be made “aware” of dialysis, it can be used to deliver appropriate cardiac support, such as to inhibit or prevent intradialytic hypotension. CFM devices can detect and treat arrhythmias, such as tachyarrhythmias, but typically cannot communicate with renal devices, such as to change dialysis treatment so as to inhibit or prevent tachyarrhythmic events (e.g., by adjusting dialysis potassium).

This document describes, among other things, examples of an apparatus or method for integrating operation of cardiac function management and renal replacement therapy devices to improve patient care, such as by using physiological parameters to determine whether a patient is at an increased risk of tachyarrhythmia and adjusting (or recommending adjustment of) the CFM device, the renal device, or both, by communicating appropriately between cardiac and renal therapy devices.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an example of portions of a cardiac and renal function management system and an environment in which it is used.

FIG. 2 illustrates an example of a method of operating the system, or a portion thereof, such as for using renal information to alter operation of a cardiac function management (CFM) device.

FIG. 3 illustrates an example of a method of operating the system, or a portion thereof, such as for using GEM device information to alter operation of a renal device.

### DETAILED DESCRIPTION

This document describes, among other things, examples of an apparatus or method for integrating operation of cardiac function management and renal replacement therapy devices to improve patient care, such as by using physiological parameters to determine whether a patient is at an increased risk of tachyarrhythmia and adjusting (or recommending adjustment of) the CFM device, the renal device, or both, by communicating appropriately between cardiac and renal therapy devices.

### System Overview Example

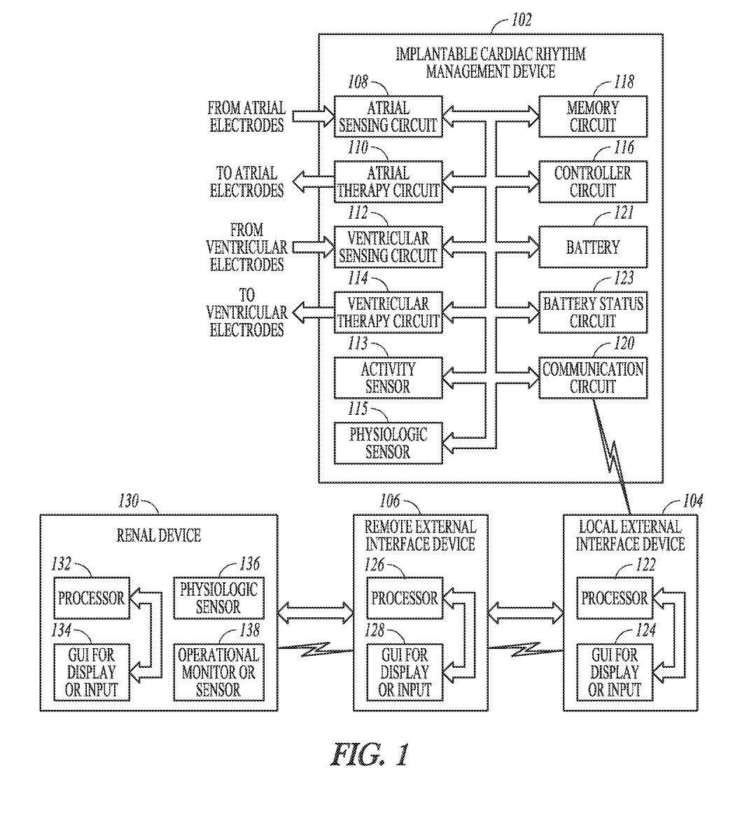

FIG. 1 shows an example of portions of a cardiac and renal function management system 100 and an environment in which it is used. In an example, the system 100 can include an ambulatory medical device, such as an external (e.g., wearable) medical device or an implantable cardiac rhythm or function management device 102, a local external interface device 104, an optional remote external interface device 106, and a renal device 130.

In an example, the implantable device 102 can include an atrial sensing circuit 108, an atrial therapy circuit 110, a ventricular sensing circuit 112, a ventricular therapy circuit 114, a controller circuit 116, a memory circuit 118, a communication circuit 120, a power source such as a battery 121, a battery status circuit 123, an activity sensor 113 configured to sense a physical activity signal of a patient or other subject, and a physiologic sensor 115 configured to sense a physiologic signal, different from the physical activity signal, of the subject.

In an example, the atrial sensing circuit 108 can be coupled to electrodes, such as an intra-atrial electrode or any other electrode that permits sensing of an intrinsic atrial cardiac signal including atrial depolarization information. The atrial therapy circuit 110 can similarly be coupled to these or other electrodes, such as for delivering pacing, cardiac resynchronization therapy (CRT), cardiac contractility modulation (CCM) therapy, defibrillation/cardioversion shocks, or other energy pulses to one or both atria.

In an example, the ventricular sensing circuit 112 can be coupled to electrodes, such as an intra-ventricular electrode or any other electrode that permits sensing of an intrinsic ventricular cardiac signal including ventricular depolarization information. The ventricular therapy circuit 114 can similarly be coupled to these or other electrodes, such as for delivering pacing, cardiac resynchronization therapy (CRT), cardiac contractility, modulation (CCM) therapy, defibrillation/cardioversion shocks, or other energy pulses to one or both ventricles.

In an example, the activity sensor 113 can include a single or multiple axis accelerometer, such as to sense an acceleration of the subject that is indicative of physical activity of the subject. The activity sensor 113 can also include a sensor interface circuit, configured to process the acceleration signal and provide a resulting physical activity signal. In an example, the physical activity signal can be indicative of a physical exertion of the subject. In an example, the activity sensor 113 can also be used for other purposes, such as to sense the subject's posture, heart sounds, or other information available from an acceleration signal.

In an example, the physiologic sensor 115 can include a respiration sensor, such as an impedance or other sensor, which can include electrodes configured to deliver a test energy, such as to the subject's thorax, and to sense a responsive voltage signal, such as indicative of the thoracic impedance, and which can be filtered to provide information about respiration, heart contraction, or thoracic fluid accumulation. In various examples, the physiologic sensor 115 can provide information about heart rate, heart rate variability, autonomic balance, heart vibrations, intracardiac pressure, thoracic fluid status, respiration, patient activity level, temperature, pH, potassium levels, oxygenation, cardiac volumes, blood pressure, or ejection fraction.

A controller circuit 116 can be coupled to the atrial sensing circuit 108 and the ventricular sensing circuit 112, such as to receive information from the sensed cardiac signals. The controller circuit 116 can also be coupled to the activity sensor 113 to receive information about the subject's physical activity or exertion level. The controller circuit 116 can also be coupled to the physiologic sensor 115, such as to receive other physiologic information. In an example, such other physiologic information can include cardiac contraction signal, such as to provide information about the subject's heart rate or interval, stroke volume, or other information available from the cardiac contraction signal. In an example, the other physiologic information can include a respiration signal, such as to provide information about the subject's breathing rate or interval, tidal volume, or other information available from the respiration signal. In an example, the controller circuit 116 can include a signal processor circuit, such as a digital signal processor (DSP) circuit, such as for extracting a template parameter from which a diagnostic indicator can be generated, as described below. In an example, the signal processor circuit can include dedicated circuitry for performing one or more signal processing functions. In an example, the controller circuit 116 can be coupled to the atrial therapy circuit 110 and the ventricular therapy circuit 114 to provide control or triggering signals to trigger timed delivery of the therapy pulses. In an example, the controller circuit 116 can be configured to provide control to help permit the CCM therapy to be effectively delivered, such as in combination with one or more other therapies (e.g., bradycardia pacing, antitachyarrhythmia pacing (ATP), cardiac resynchronization therapy (CRT), atrial or ventricular defibrillation shock therapy) or functionalities (e.g., autothreshold functionality for automatically determining pacing threshold energy, autocapture functionality for automatically adjusting pacing energy to capture the heart, etc.). In an example, this can include providing dedicated modules within the controller circuit 116, or providing executable, interpretable, or otherwise performable code configure the controller circuit 116.

A memory circuit 118 is coupled to the controller circuit 116, such as to store control parameter values, physiological data, or other information. A communication circuit 120 is coupled to the controller circuit 116 to permit radiofrequency (RF) or other wireless communication with an external device, such as the local external interface device 104 or the remote external interface device 106.

In an example, the battery 121 can include one or more batteries to provide power for the implantable device 102. In an example, the battery 121 can be rechargeable, such as by wireless transcutaneous power transmission from an external device to the implantable device 102. The battery status circuit 123 can be communicatively coupled to each of the battery 121 and the controller circuit 116, such as to determine battery status information, for example, indicative of how much energy remains stored in the battery 121. The controller circuit 116 can be configured to alter operation of the implantable device 102, such as based at least in part on the battery status information.

In an example, the local external interface device 104 can include a processor circuit 122 and a graphic user interface (GUI) 124 or like device for displaying information or receiving user input as well as a communication circuit, such as to permit wired or wireless communication with the remote external interface device 106 over a communications or computer network. Similarly, the remote external interface device 106 can include a processor circuit 126 and a graphic user interface (GUI) 128 or like device for displaying information or receiving user input as well as a communication circuit, such as to permit wired or wireless communication with the local external interface device 104 over the communications or computer network.

Because the system 100 includes processing capability in the ambulatory or implantable device 102 (e.g., provided by the controller circuit 116), the local external interface device 104 (e.g., provided by the processor 122), and the remote external interface device 106 (e.g., provided by the processor 126), various methods discussed in this document can be implemented at any of such locations, or tasks can be distributed between two or more of such locations.

The system 100 can also include a renal device 130. The renal device 130 can include a processor circuit 132 and a graphic user interface (GUI) 134 or like device for displaying information or receiving user input. The renal device 130 can also include a communication circuit, such as to permit wired or wireless communication with one or more of the remote external interface device 106, the local external interface device 104, or the implantable cardiac function management device 102. Such communication can include communication over a communications or computer network.

The renal device 130 can include a renal device, such as for providing renal therapy, such as one or more of hemodialysis, peritoneal dialysis, hemofiltration, hemodiafiltration, intestinal dialysis, or other renal therapy. The renal device 130 can include a wearable, implantable, or other ambulatory renal device 130, or an external non-ambulatory renal device 130, such as an external kidney dialysis machine that can be configured to provide treatment to a dialysis patient at a patient station in a clinical or home setting.

In an example, the renal device 130 can include a sorbent device, which can include activated charcoal, urease, zirconium phosphate, hydrous zirconium oxide, or activated carbon. In an example, the renal device 130 can provide a therapeutic agent, such as potassium, a polypeptide or variant thereof that can prevent, inhibit, delay, or alleviate loss of renal function, or an anticoagulant, such as heparin, prostacyclin, hirudin, or sodium citrate, or other pharmaceutical, biological, or other therapeutic agent.

The CFM device 102 or the renal device 130 can be configured to gather and share (e.g., one with the other) physiological information, device operational information, or other information automatically, or by intervention from a user, such as by a caregiver or the patient. The remote external interface device 106 can be configured to store, process, or transmit such information, or other information derived therefrom, such as via a wired or wireless signal, such as via a computer or telecommunications or other network. Information can be communicated between the CFM device 102 and the renal device 130, such as via the local external interface device 104 or the remote external interface device 106, or directly.

In an example, physiologic information about the subject gathered by the CFM device 102 and shared with the renal device 130 can include information obtained or derived from the physiologic sensor 115, the activity sensor 113, the atrial sensing circuit 108, or the ventricular sensing circuit 112, such as described above. In an example, such physiologic information gathered by the GEM device 102 to be shared with the renal device 130 can include information about one or more indications of a detected or predicted tachyarrhythmia or a vulnerability to such a tachyarrhythmia.

In an example, the renal device 130 can include one or more physiological sensors 136 from which physiologic information about the subject can be gathered. Such physiologic information, or information derived from such physiologic information obtained by the renal device 130, can be shared with the CFM device 102. Examples of such physiologic sensors 136 that can be included in or coupled to the renal device 130 can include a blood sensor (e.g., such as for sensing blood hematocrit, oxygenation, creatinine, blood urea nitrogen (BUN), albumin, potassium, sodium, calcium, phosphorus, pH, electrolytes, glucose, or one or more other blood constituents), or a dialysate sensor (e.g. such as dialysate BUN, creatinine, electrolytes), or patient monitoring sensors (e.g. such as for sensing access blood flow, central blood volume, cardiac output, whole body bioimpedance, blood pressure). In an example, such physiologic information to be shared with the renal device 130 can include information about one or more indications of a detected or predicted tachyarrhythmia or of a vulnerability to such a tachyarrhythmia.

In an example, device operational information (or information derived from such device operational information) gathered by the CFM device 102 and shared directly or indirectly with the renal device 130 can include information obtained or derived from the battery status circuit 123, the controller circuit 116, the memory circuit 118, the atrial therapy circuit 110, the ventricular therapy circuit 114, or the physiologic sensor 115 such as described above. In an example, such device operational information to be shared by the GEM device 102 with the renal device 130 can include information about one or more indications of a detected or predicted tachyarrhythmia or of a vulnerability to such a tachyarrhythmia. In an example, such device operational information to be shared by the CFM device 102 with the renal device 130 can include information about one or more indications of a device response of the CFM device 102 to a detected or predicted tachyarrhythmia or of a vulnerability to such a tachyarrhythmia (e.g., scheduling or delivery of antitachyarrhythmia pacing (ATP) or defibrillation shock, or adjusted or enhanced tachyarrhythmia monitoring by the CFM device 102).

In an example, device operational information (or information derived from such device operational information) obtained by the renal device 130, can shared directly or indirectly by the renal device 130 with the GEM device 102. The renal device 130 can include one or more operational status monitors or sensors 138, which can include one or more of a flow monitor or sensor (e.g., ultrafiltration rate, clearance, time on dialysis, blood flow, dialysate flow), a pressure sensor (e.g., blood pressure, dialysate pressure), therapeutic agent sensor hematocrit, central blood volume), or the like. In an example, such device operational information obtained by the renal device 130 to be shared with the CFM device 102 can include information about one or more indications of a detected or predicted tachyarrhythmia or a vulnerability to such a tachyarrhythmia (e.g., such as induced by or monitored during a currently ongoing (e.g., intradialytic) dialysis or other renal therapy episode, or during or between one or more preceding (e.g., interdialytic) dialysis or other renal therapy episodes.

### Example of Method of Triggering CFM Operation Using Renal Information

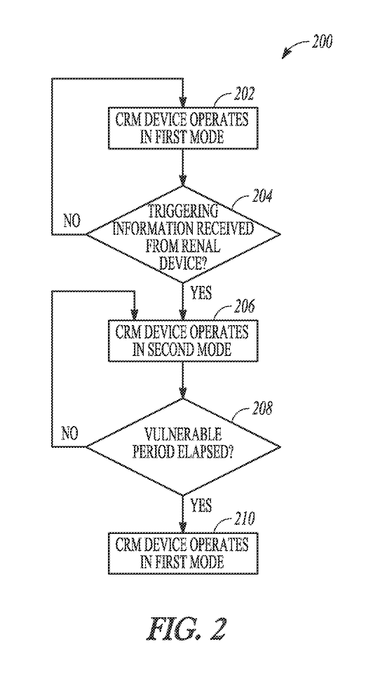

FIG. 2 illustrates an example of a method 200 of operating the system 100, or a portion thereof. At 202, the CFM device 102 can be operating in a first operating mode. This first operating mode can include a normal operating mode of the CFM device 102. Such normal first operating mode need not account for information about or from the renal device 130; in such mode, the CFM device 102 otherwise provides CFM monitoring or therapy without information about or from the renal device 130. At 204, triggering information can be received directly or indirectly by the CFM device 102. Such triggering information can be about or from the renal device 130. Such triggering information can be pertinent to CFM operation, such as by being indicative of a period of increased vulnerability to tachyarrhythmia. In an example, the period of increased vulnerability to tachyarrhythmia can include a vulnerable period due to ongoing renal therapy then being delivered by the renal device 130, such as an intradialytic period associated with then-occurring dialysis. In an example, the vulnerable period can include an interdialytic period associated with previously-delivered renal therapy, the delivery of which has elapsed, but for which a period of increased tachyarrhythmia vulnerability of the subject remains (e.g., for up to a specified time (e.g., between 3-5 hours (e.g., 4 hours) after cessation of the previous dialysis session) or other condition that serves as an indicator that the patient has stabilized from the previously-delivered renal therapy).

At 206, upon receiving the triggering information about or from the renal device 130, the GEM device 102 can switch from operating in the first operating mode to operating in a different second operating mode. In an example, the second operating mode can be configured to be appropriate for use during a period of increased vulnerability to a tachyarrhythmia, such as an intradialytic period or an interdialytic period. At 208, when the vulnerable period has elapsed (e.g., timed-out or cancelled on the basis of updated triggering information about or from the renal device 130), then at 210 the CFM device 102 can resume operating in the first operating mode, otherwise process flow can return to 206, and the CFM device 102 can continue operating in the second operating mode.

### Examples of Triggering Information about or from the Renal Device

Some illustrative examples of information about or from the renal device 130 that can be used to switch the CFM device 102 from operating in the first operating mode to the second operating mode can include one or more of the following.

1. Ongoing Dialysis or Other Renal Therapy has been Initiated.

This can include triggering a CFM device 102 operating mode switch from the first operating mode to the second operating mode either upon renal therapy initiation or during renal therapy after a specified delay from initiating renal therapy. This renal-related triggering information can be received by the CFM device 102 automatically, such as in response to a direct or indirect communication generated by the renal device 130, or in response to information about the renal device 130 generated or entered manually into one or more locations of the system 100 by a caregiver, and automatically or manually communicated to the CFM device 102, either directly or indirectly. Initiating dialysis, ultrafiltration, or other renal therapy by the renal device 130 can increase a vulnerability of the subject to tachyarrhythmia during or for a period after the renal therapy.

2. Ongoing Dialysis or Other Renal Therapy has been Adjusted.

This can include triggering a CFM device 102 operating mode switch from the first operating mode to the second operating mode upon or at a specified delay after the renal therapy of the renal device 130 has been changed, such as by adjusting renal therapy flow rate, dialysate composition, therapeutic agent administration or the like. Adjusting dialysis or other renal therapy can alter the vulnerability of the subject to tachyarrhythmia during the renal therapy, or for some period of time that follows cessation of such renal therapy.

3. Dialysis Session has been Completed.

This can include triggering a CFM device 102 operating mode switch from the first operating mode to the second operating mode either upon renal therapy initiation, during renal therapy after a specified delay from initiating renal therapy, upon completion of the renal therapy, or after a specified delay from completing the renal therapy. This renal-related triggering information can be received by the CFM device 102 automatically, such as in response to a direct or indirect communication generated by the renal device 130, or in response to information about the renal device 130 generated or entered manually into one or more locations of the system 100 by a caregiver, and automatically or manually communicated to the CFM device 102, either directly or indirectly. Ceasing dialysis or other renal therapy can initiate or prolong a period of vulnerability of the subject to tachyarrhythmia during or for a period after the renal therapy, and the nature of such tachyarrhythmia vulnerability may be similar to or different from the nature of such tachyarrhythmia vulnerability during the renal therapy.

4. Ongoing or Previous Renal Monitoring Indicates a Physiologic Condition.

This can include detecting, such as by using the renal device 139, an indication of a physiological condition that can indicate an increased vulnerability to tachyarrhythmia, such as an electrolyte imbalance, poor blood oxygenation, a blood chemical indication of sympathetic overassertion in a sympathovagal autonomic balance, or the like. Such detection can occur during renal monitoring by the renal device 130, such as during an ongoing dialysis or other renal therapy session, or during a most recent dialysis or other renal therapy session, or during a more previous dialysis or other renal therapy session, or during a renal monitoring session that did not include providing dialysis or other renal therapy.

### Examples of Renal-Responsive Second Operating Mode of the CFM Device

1. Calibration or Self-Check.

In an example, the CFM device 102 can perform a calibration or self-check function to ensure that such CFM device 102 is performing correctly, such as based on a triggering communication received about or from the renal device 130.

In an example, the physiologic sensor 115 of the CFM device 102 can include a thoracic impedance sensor, such as to compute fluid status for determining pulmonary edema, pleural effusion, hypotension, or to determine minute ventilation (MV), such as for providing an indicator of the metabolic need of the subject thr controlling a pacing rate of the subject. The fluid status or other information measured using thoracic impedance may be affected by fluid being removed or added by the renal device 130, such as during dialysis or ultrafiltration.

Accordingly, such information about the renal device 130 can be used to trigger calibration of the thoracic impedance sensor of the CFM device 102. Such information about the renal device 130 can be used to adjust a threshold value to which such raw or signal-processed impedance is compared, such as to declare an abnormal fluid status condition (such as pulmonary edema, or a congestive heart failure decompensation or other CHF status indication that is based at least in part upon detection of such an abnormal fluid status condition). For example, during dialysis, the thoracic impedance can be compared to a more stringent (or less stringent) threshold value for declaring a “wet” (hypervolemic) status. This can help inhibit or prevent false positive or false negative volume status indications.

2. Qualify or Modify Use or Logging of Physiologic Information.

In an example, physiologic information obtained by the CFM device 102 during or after renal therapy being delivered by the renal device 130 can be qualified or modified in response to triggering information received from the renal device 130.
In an example, trending of thoracic impedance information used for fluid status determination can flag or disregard data obtained during or immediately following a dialysis or other renal therapy session. This can help reduce or avoid the contribution of known dialysis-induced fluctuations in such data into the patient's fluid status determination. Similar techniques can be applied to other data that can be affected by a dialysis or other renal therapy session.
In an example, a threshold value used by the CFM device 102 to generate an alert condition can be modified in response to triggering information received from the renal device.

3. Adjust CFM Therapy being Delivered.

In an example, CFM therapy delivered by the CFM device 102 can be adjusted, such as in the second operating mode, with respect to the first operating mode, such as in response to triggering information about or from the renal device 130 indicating an ongoing or previous renal therapy session carried out by the renal device 130, or in response to physiological or device operating triggering information about or from the renal device 130 that can indicate a need to adjust the CFM therapy.

For example, dialysis or other renal therapy can cause or increase the potential for hypotension in the subject. Accordingly, in response to triggering information about or from the renal device 130 that dialysis or other renal therapy is ongoing or has just been completed or the like, the CFM device 102 can adjust CFM therapy being delivered, such as to increase cardiac output to inhibit or prevent hypotension. Illustrative examples of adjusting CFM therapy in the second operating mode can include:
increasing pacing rate, such as by a specified increment above a then-indicated pacing rate computed from a physical activity sensor, a minute ventilation (MV) sensor, or other indication of metabolic need of the subject;
increasing stimulation during a cardiac refractory period to increase cardiac contractility and cardiac output;
modifying neuromodulation of cardiac or systemic afferent or efferent nerves, baroreceptors, or associated ganglia (for example, pulmonary artery pressure receptors, cardiopulmonary baroreceptors) to increase cardiac output and/or systemic vascular resistance;
increasing electrostimulation energy, such as by a specified amount, to ensure capture;
adjusting electrostimulation electrode configuration, such as from single-chamber ventricular pacing to biventricular or dual chamber pacing, so as to increase spatial coordination;
adjusting an interelectrode delay between same-chamber (e.g., between multiple left-ventricular electrodes on a quadripolar coronary sinus or other multi-electrode lead) or different chamber (e.g., atrioventricular (AV) delay, bi-ventricular (RV-LV delay), etc.) electrostimulations delivered during the same cardiac cycle;
initiate or adjust a His-bundle pacing, such as for providing bi-ventricular coordination of ventricular heart contractions from a right ventricular (RV) septal location at or near the His-bundle, at which an electrostimulation delivered; or
adjusting anti-tachyarrhythmia therapy, such as by changing the number of high-rate or morphologically-abnormal beats needed to declare detection of a tachyarrhythmia episode to discriminate between different types of tachyarrhythmia beats, or by changing a response to a detected tachyarrhythmia, such as to increase or decrease the likelihood of delivering ATP vs. a shock, or the shock energy.

4. Logging or Learning.

In an example, the CFM device 102 can respond to the triggering information provided by the renal device 130 by storing the triggering information or other physiologic or device operating information provided by the renal device 130, such as in conjunction with information from the CFM device 102, either at the CFM device 102, at the remote external interface device 106, at the local external interface device 104, or at the renal device 130. This can allow information from both the GEM device 102 and the renal device 130 to be used together, such as to adjust or coordinate current or future operation of one or both of the CFM device 102 or the renal device 130.

For example, physiologic or device operational parameters provided by the renal device 130 during a dialysis or other renal therapy session, or within a specified time following such a renal therapy session, can include one or more parameters that can be indicative of a tachyarrhythmia vulnerability (e.g., electrolyte imbalance, renal therapy flow rate or composition), which can be stored in conjunction with the CFM physiologic or device operational parameters, such as for altering (either automatically, or by suggesting such modification to a physician or other caregiver) current or future CFM therapy. Over time, such information can yield information about a threshold for inducing tachyarrhythmia vulnerability via the renal therapy, and the CFM therapy can be configured to avoid such threshold of tachyarrhythmia vulnerability, such as during or after such a renal therapy session.

### Example of Method of Triggering Renal Device Operation Using Information about or from a CFM Device

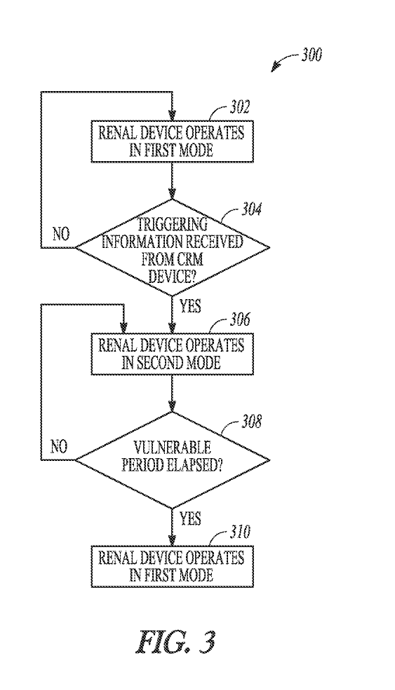

FIG. 3 illustrates an example of a method 300 of operating the system 100, or a portion thereof. At 302, the renal device 130 can be operating in a first operating mode. This first operating mode can include a normal operating mode of the renal device 130. Such normal first operating mode need not account for information about or from the CFM device 102; in such mode, the renal device 130 otherwise provides renal monitoring or therapy without information about or from the CFM device 102. At 304, triggering information can be received directly or indirectly by the renal device 130. Such triggering information can be about or from the CFM device 102. Such triggering information can be pertinent to renal device 130 operation, such as by being indicative of a period of increased vulnerability to tachyarrhythmia. In an example, the period of increased vulnerability to tachyarrhythmia can include a vulnerable period detected or determined by the CFM device 102, such as a previous or current indication of a detected or predicted tachyarrhythmia.

At 306, upon receiving the triggering information about or from the CFM device 102, the renal device 130 can switch from operating in the first operating mode to operating in a different second operating mode. In an example, the second operating mode can be configured to be appropriate for use during a period of increased vulnerability to a tachyarrhythmia, such as indicated by the CFM device 102. At 308, when the vulnerable period has elapsed (e.g., timed-out or cancelled on the basis of updated triggering information about or from the CFM device 102), then at 310 the renal device 130 can resume operating in the first operating mode, otherwise process flow can return to 306, and the renal device 130 can continue operating in the second operating mode.

### Examples of Triggering Information about or from the CFM Device

Some illustrative examples of information about or from the CFM device 102 that can be used to switch the renal device 130 from operating in the first operating mode to the second operating mode can include one or more of the following.

1. Heart Rate, Heart Rate Variability, Premature Ventricular or Atrial Contraction (PVC, PAC), Long-Short Sequence, or Prolonged Qt-Interval Indications of Tachyarrhythmia Vulnerability.

In an example, the CFM device 102 can be configured to detect heart contractions, such as from an intrinsic cardiac signal obtained from the atrial sensing circuit 108 or the ventricular sensing circuit 112, or from the physiologic sensor 115 (e.g., an impedance-based cardiac contraction signal, or a mechanical or fluid-based cardiac contraction signal). From such detected heart contractions, an intrinsic heart rate can be determined, such as by the controller circuit 116. Further, a heart rate variability (HRV) indication can also be determined, such as from time intervals between the detected heart contractions, by the controller circuit 116. The HRV can be trended over time. A decrease in HRV can indicate sympathetic over-assertion in the sympathovagal balance of the autonomic nervous system of the subject. Such decreased HRV, therefore, can indicate an increased vulnerability to tachyarrhythmia. Similarly, the occurrence, count, or frequency of PVC/PACs can indicate an increased vulnerability to tachyarrhythmia, particularly when combined with other physiologic information, such as the physical activity of the subject. For example, post-exercise PVC/PACs can be particularly indicative of a tachyarrhythmia vulnerability. Moreover, the occurrence, count, or frequency of long-short sequences of temporally adjacent intervals between temporally adjacent heart contractions can also be indicative of a tachyarrhythmia vulnerability. Furthermore, the occurrence, count, or frequency of prolonged rate-adjusted QT intervals can also be indicative of a tachyarrhythmia vulnerability. Still further, the occurrence, count, or frequency of short RR intervals between ventricular contractions (e.g., high intrinsic contraction rate, such as in the absence of accompanying physical activity) or abnormal-morphology beats can also be indicative of a tachyarrhythmia vulnerability or an ongoing tachyarrhythmia.

Information about HRV, about a decrease in HRV corresponding to an increased risk of tachyarrhythmia, about the occurrence or prevalence of PVC/PACs indicative of tachyarrhythmia vulnerability, about the occurrence or prevalence of long-short sequences of adjacent heart contraction intervals, about the occurrence or prevalence of rate-adjusted prolonged QT intervals, or about high rate of heart contractions or abnormal-morphology beats, can be communicated from the CFM device 192 to the renal device 139, such as to provide triggering information to permit the renal device 130 to adjust its operation accordingly during or after such tachyarrhythmia vulnerable period, such as to reduce the risk of renal therapy being delivered by the renal device 130 from inducing or exacerbating a tachyarrhythmia in the subject.

2. Thoracic Fluid, Heart Sound, Pressure Indication.

In an example, the CFM device 102 can include one or more physiologic sensors 115 that can include one or more of a thoracic fluid sensor, heart sound sensor, or pulmonary artery pressure (PAP) sensor, which can provide information that can be used for determining fluid status. Such sensors can be used to determine the fluid state of the patient during and following dialysis therapy. Such information can be provided by the CFM device 102 to the renal device 130, such as to provide triggering information to initiate, adjust, or titrate ultrafiltration, or other renal therapy delivered by the renal device 130. In an example, increased thoracic impedance corresponds to decreased thoracic fluid accumulation and therefore thoracic impedance determined be the CFM device 102 can be used to indicate a vulnerability of the subject to arrhythmias or hypotension in response to thoracic fluid status. Information about the subject's fluid status or increased vulnerability to tachyarrhythmia can be communicated from the CFM device 102 to the renal device 130, such as to provide triggering information to initiate, adjust, or titrate dialysis, ultrafiltration, or other renal therapy.

3. Chemical Sensor Indication.

In an example, the CFM device 102 can include a physiologic sensor 115 that can include a chemical sensor, such as for detecting the amount of potassium, calcium, or another substance in the subject's bloodstream. Information from such chemical sensor included in or coupled to the CFM device 102 can be provided by the CFM device 102 to the renal device 130, such as to provide triggering information to initiate, adjust, or titrate dialysis, ultrafiltration, infusion (e.g., which can include administration of potassium, calcium, or one or more other chemical constituents), or other renal therapy delivered by the renal device 130. In an example, chemical information determined by the CFM device 102 can be used to indicate a heightened vulnerability of the subject to tachyarrhythmia, such as can exist when there are significant changes in serum potassium and other electrolytes during or immediately following dialysis indicative of an increased vulnerability to tachyarrhythmia. Information about the potassium level or other chemical information indicating increased vulnerability to tachyarrhythmia can be communicated from the CFM device 102 to the renal device 130, such as to provide triggering information to initiate, adjust, or titrate ultrafiltration, or other renal therapy.

4. Renal Status or Renal Therapy Status.

In an example, the CFM device 102 can indirectly detect renal status or renal therapy status information, such as the onset or termination of renal therapy, such as ultrafiltration. For example, ultrafiltration-related hemoconcentration can be detected using a blood conductivity characteristic sensor, such as a blood impedance sensor. In an illustrative example, the blood impedance sensor can include two electrodes that can be located on a distal portion of an intravascular lead that can be included in or coupled to the CFM device 102. More than two electrodes can be used to perform such impedance sensing, such as to implement a three-point or a four-point probe for performing such blood impedance sensing or other blood conductivity characteristic sensing (e.g., voltage sensing, transconductance sensing, transimpedance sensing, or other blood conductivity characteristic sensing). The electrodes can be, but need not both be, located within the same blood vessel or heart chamber. One or more other components of the sensed conductivity characteristic signal can be filtered or otherwise signal processed, such as to remove a respiration component of an impedance signal, a cardiac stroke component of the impedance signal, or the like.

In an example of such indirect sensing of a renal therapy parameter using the CFM device 102, an increase in blood impedance can indicate an increased hemoconcentration (which, in turn, can indicate onset of ultrafiltration) and a decrease in blood impedance can indicate a decreased hemoconcentration (which, in turn, can indicate termination of ultrafiltration).

Such indirect sensing of a renal therapy parameter can also use one or more other physiological or other signals. For example, information about the sensed blood conductivity characteristic can be combined with information about the time of day, information about the subject's posture (e.g., sensed using a 3-axis accelerometer or other posture sensor that can be included in the CFM device 102). For example, the sensed blood conductivity characteristic information can be combined with one or more of the time of day or the posture information to compensate for one or both such effects on the blood conductivity characteristic or the hemoconductivity, such that a better correlation between sensed blood conductivity information and hemoconcentation or other indirect renal information can be determined with better sensitivity, specificity, or both.

### Examples of CFM-Responsive Second Operating Mode of the Renal Device

1. Dialysis or Ultrafiltration.

In an example, the renal device 130 can respond to the triggering information provided by the CFM device 102 by initiating, adjusting, or titrating dialysis or ultrafiltration. For example, a fluid-accumulation indication provided by the CFM device 102 can be responded to by initiating or increasing dialysis or ultrafiltration by the renal device 130. In an example, responsive to triggering information from the CFM device 102 indicating a tachyarrhythmia vulnerability (e.g., decreased HRV, thoracic fluid accumulation, electrolyte imbalance or other blood chemical information, reduced blood flow, or the like), the renal device 130 can respond by initiating, adjusting, or titrating dialysis, ultrafiltration, or composition of the dialysate.

2. Infusion.

In an example, the renal device 130 can respond to the triggering information provided by the GEM device 102 by initiating, adjusting, or titrating infusion, such as responsive to such triggering information indicating a tachyarrhythmia episode or intradialytic hypotension (e.g., from PAP, heart sound, thoracic impedance-derived fluid accumulation, or other indication of CHF status such as CHF decompensation). For example, a fluid-accumulation indication provided by the CFM device 102 can be responded to by initiating or increasing infusion of a therapeutic agent such as a plasma expander by the renal device 130. In an example, responsive to triggering information from the CFM device 102 indicating a tachyarrhythmia vulnerability (e.g., decreased HRV, impedance-indicated fluid accumulation, electrolyte imbalance or other blood chemical information, reduced blood flow, or the like), the renal device 130 can respond by initiating, adjusting, or titrating infusion, such as by ceasing or reducing infusion of the therapeutic agent or that of an electrolyte causing an electrolytic imbalance, or by initiating or increasing infusion of an anti-tachyarrhythmic drug, a balancing electrolyte, or other therapeutic agent.

3. Dialysis.

In an example, the renal device 130 can respond to the triggering information provided by the CFM device 102 by initiating, adjusting, or titrating dialysis, such as responsive to such triggering (e.g., from PAP, heart sound, thoracic impedance-derived fluid accumulation). For example, a fluid-accumulation indication provided by the CFM device 102 can be responded to by initiating or modifying the therapy offered by renal device 130, such as within a dialysate during dialysis. In an example, responsive to triggering information from the CFM device 102 indicating a tachyarrhythmia vulnerability (e.g., decreased HRV, impedance-indicated hypotension, electrolyte imbalance or other blood chemical information, reduced blood flow, or the like), the renal device 130 can respond, such as by:
initiating, adjusting, or titrating dialysis, such as by ceasing or reducing dialysate infusion of a diuretic agent or that of an electrolyte causing an electrolytic imbalance;
initiating or increasing dialysate infusion of an anti-tachyarrhythmic drug, a balancing electrolyte, or other therapeutic agent;
reducing or ceasing dialysis, or an intradialytic rate at which such dialysis is being provided, or by increasing an interdialytic interval between dialysis sessions; or
adjusting a parameter of a subsequent dialysis or other renal therapy session.

4. Logging or Learning.

In an example, the renal device 130 can respond to the triggering information provided by the CFM device 102 by storing the triggering information or other physiologic or device operating information provided by the CFM device 102, such as in conjunction with information from the renal device 130, either at the renal device 130, at the remote external interface device 106, at the local external interface device 104, or at the CFM device 102. This can allow information from both the CFM device 102 and the renal device 130 to be used together, such as to adjust or coordinate current or future operation of one or both of the CFM device 102 or the renal device 130.

For example, physiologic parameters sensed by the CFM device 102 during a dialysis or other renal therapy session, or within a specified time following such a renal therapy session, can include one or more parameters that can be indicative of a tachyarrhythmia vulnerability (e.g., heart rate, HRV, prolonged QT interval, PVCs, long-short contraction sequences, heart sound indicating changes in volume status or contractility, pressure indicating changes in volume status, thoracic impedance indicating changes in thoracic or body fluid status, blood analyte indicating electrolyte imbalance or poor blood oxygenation), which can be stored in conjunction with the particular dialysis or other renal therapy parameters, such as for altering (either automatically, or by suggesting such modification to a physician or other caregiver) a subsequent renal therapy session. Over time, such information can yield information about a threshold for inducing tachyarrhythmia vulnerability via the renal therapy, and the renal therapy can be configured to avoid such threshold of tachyarrhythmia vulnerability.

### Examples of Triggering Information about or from Both the CFM Device and the Renal Device

The renal device 130 and the CFM device 102 can jointly provide physiological or other information, such as can be used as triggering information at 204 or 304. For example, the triggering information received from the renal device 130 at 204 can include information generated by the renal device 130 in conjunction or cooperation with the CFM device 102, or the triggering information received from the CFM device 102 at 304 can include information generated by the CFM device 102 in conjunction or cooperation with the renal device 130.

An illustrative example of cooperation between the CFM device 102 and the renal device 130 can include or use an extracorporeal blood circuit, through which the renal device 130 is coupled to a patient's circulatory system, such as for performing hemodialysis, hemofiltration, hemodiafiltration, ultrafiltration, or the like. The renal device 130 can modify a parameter (e.g., blood temperature, or blood conductivity), which can be detected using a CFM device 102 (e.g., using a thermodilution technique to detect a change in blood temperature at a temperature sensor that can be located on a distal or other portion of an intravascular lead that can be included in or coupled to the CFM device 102).

In such a thermodilution example, the renal device 130 can include a blood or dialysate heater or cooler that can heat or cool a bolus of blood or dialysate passing through the extracorporeal blood circuit. The CFM device 102 can include a temperature sensor, such as can be located on a distal or other portion of an intravascular lead that can be included in or coupled to the CFM device 102. A transit time between (1) the location in the extracorporeal blood circuit at which the blood temperature was modified by the renal device 130 and (2) the location at which blood temperature or change in blood temperature is detected using the CFM device 102 can be measured, such as for use in calculating one or more physiological parameters, such as blood flow velocity, cardiac output, central blood volume, or any combination thereof, or for calculating another physiological or other parameter of interest.

In a conductivity modulation example, the renal device 130 can include a blood conductivity modulator, such as a dialysate or other infusate pump or other dispensing device. The blood conductivity can be modulated by adjusting one or more of the volume of dialysate or other infusate introduced by the blood conductivity modulator, or the content of the solution being infused. For example, hypertonic saline can be introduced, by itself, or mixed with another dialysate or other infusate, to adjust the conductivity of a bolus of blood passing through the extracorporeal blood circuit to which the renal device 130 is coupled. The CFM device 102 can include an impedance or other blood conductivity characteristic detector (e.g., such as described herein, using electrodes located on a distal or other portion of an intravascular lead) that can detect the arrival of a conductivity-modulated bolus of blood. A transit time between (1) the location in the extracorporeal blood circuit at which the blood conductivity was modified by the conductivity modulator device component of the renal device 130 and (2) the location at which blood conductivity or change in blood conductivity is detected using the CFM device 102 can be measured, such as for use in calculating one or more physiological parameters, such as blood flow velocity, cardiac output, central blood volume, or any combination thereof, or for calculating another physiological or other parameter of interest.

### Additional Notes and Examples

Although the above examples have discussed operational changes in at least one of the CFM device 102 or the renal device 130 in response to triggering information obtained from the other of the CFM device 102 or the renal device 130, such operational changes need not be automatic, as described above. Instead, such operational changes can involve providing an alert or recommendation to a human, such as the subject or a caregiver, who can then provide an instruction to direct or confirm such an operational change.

Example 1 can include subject matter (such as an system, apparatus, method, tangible machine readable medium, etc.) that can include a wearable or implantable ambulatory cardiac function management (CFM) device. The CFM device can include a data input configured to receive information about or from an external renal monitoring or therapy device. The subject matter can include first and second operating modes, such as of a control circuit. The first operating mode can control operation of the implantable cardiac function management device in the absence of triggering information about or from the renal monitoring or therapy device. The second operating mode can control operation of the implantable cardiac function management device in response to receiving triggering information about or from the renal monitoring or therapy device. The second operating mode can include an exit condition, which can return control of operation of the implantable cardiac function management device to the first operating mode, when a vulnerable period has elapsed. The vulnerable period can include a time period corresponding to an increased risk of tachyarrhythmia or intradialytic hypotension.

In Example 2, the subject matter of Example 1 can include the control circuit being configured to adjust or direct therapy, provided by the cardiac function management device, in response to the triggering information received from the renal monitoring or therapy device.

In Example 3, the subject matter of any one of Examples 1-2 can include the control circuit being configured to adjust or direct an anti-tachyarrhythmia therapy, provided by the cardiac function management device, in response to the triggering information received from the renal monitoring or therapy device.

Example 4, the subject matter of any one of Examples 1-3 can include the control circuit being configured to adjust or direct a physiological sensor of the cardiac function management device in response to the triggering information received from the renal monitoring or therapy device.

In Example 5, the subject matter of any one of Examples 1-4 can include the control circuit being configured to qualify or flag data from the physiological sensor of the cardiac function management device using the triggering information received from the renal monitoring or therapy device.

In Example 6, the subject matter of any one of Examples 1-5 can include the control circuit being configured to adjust determination of a thoracic fluid accumulation status or a congestive heart failure (CHF) status in response to the triggering information received from the renal monitoring or therapy device.

In Example 7, the subject matter of any one of Examples 1-6 can include the operating mode of the cardiac function management device being adjusted in response to the triggering information received from the renal monitoring or therapy device, wherein the triggering information indicates at least one of: (1) initiation of renal therapy; (2) adjustment of ongoing renal therapy; (3) cessation of renal therapy; or (4) a physiologic condition indicated by the renal monitoring or therapy device.

In Example 8, the subject matter of any one of Examples 1-7 can include the second operating mode of the cardiac function management device adjusting, with respect to the first operating mode, at least one of pacing rate, electrostimulation energy, electrostimulation electrode configuration, interelectrode electrostimulation delay, His-bundle pacing, or anti-tachyarrhythmia therapy.

In Example 9, the subject matter of any one of Examples 1-8 can include logging (e.g., at the GEM device) information received by the CFM device about or from an external renal monitoring or therapy device.

Example 10 can include, or can be combined with any one of Examples 1-9 to include, subject matter (such as an system, apparatus, method, tangible machine readable medium, etc.) such as can include or use an apparatus comprising a renal monitoring or therapy device. The renal monitoring or therapy device can include a tachyarrhythmia vulnerability detection circuit, which can be configured to monitor a patient physiological or device operational renal parameter that varies during or in response to renal therapy. The renal monitoring or therapy device can be configured to determine whether the renal parameter indicates a vulnerable period corresponding to an increased risk of a present or future tachyarrhythmia episode. The renal monitoring or therapy device can include a communication circuit, configured to communicate directly or indirectly to an ambulatory cardiac function management device to provide a tachyarrhythmia vulnerability mode trigger configured to alter operation of the cardiac function management device in response to the trigger.

In Example 11, the subject matter of any one of Examples 1-10 can comprise the renal monitoring or therapy device including a control circuit that is configured to direct therapy provided by the renal monitoring or therapy device in response to information received from the cardiac function management device about at least one of a tachyarrhythmia vulnerability of the subject, a fluid accumulation status of the subject, or a heart failure status of the subject.

In Example 12, the subject matter of any one of Examples 1-11 can include the renal monitoring or therapy device being configured to adjust or direct an anti-tachyarrhythmia therapy, provided by the cardiac function management device.

In Example 13, the subject matter of any one of Examples 1-12 can include the renal monitoring or therapy device being configured to adjust determination of a thoracic fluid accumulation status or a congestive heart failure (CHF) status by the cardiac function management device.

In Example 14, the subject matter of any one of Examples 1-13 can include the renal monitoring or therapy device being configured to adjust an operating mode of the cardiac function management device using information about at least one of: (1) initiation of renal therapy; (2) adjustment of ongoing renal therapy; (3) cessation of renal therapy; or (4) a physiologic condition indicated by the renal monitoring or therapy device.

In Example 15, the subject matter of any one of Examples 1-14 can include the control circuit of the renal monitoring or therapy device being configured to adjust or direct the renal monitoring therapy, in response to the information received from the cardiac function management device, by providing a control signal to modify at least one of the following: dialysis, blood flow rate, dialysate flow rate, ultrafiltration, dialysate composition, or infusion.

In Example 16, the subject matter of any one of Examples 1-15 can include logging (e.g., at the renal monitoring or therapy device) information received from or about the cardiac function management device.

In Example 17, the subject matter of any one of Examples 1-16 can include altering operation of the renal monitoring or therapy device in response to information received from or about the cardiac function management device.

Example 18 can include, or can be combined with any one of Examples 1-17 to include, subject matter (such as an system, apparatus, method, tangible machine readable medium, etc.) such as can include an implantable or wearable ambulatory cardiac function management device comprising a tachyarrhythmia detection or prediction circuit, which can be configured to detect the presence of an existing or a predicted future tachyarrhythmia episode. The cardiac function management device can include a communication circuit, configured to communicate directly or indirectly to an external renal monitoring or therapy device triggering information that is configured to alter operation of the renal monitoring or therapy in response to the triggering information.

In Example 19, the subject matter of any one of Examples 1-18 can include the communication circuit being configured to communicate directly or indirectly to an external renal monitoring or therapy device triggering information indicating a tachyarrhythmia vulnerability including at least one of: heart rate, heart rate variability, premature ventricular contraction information, long-short contraction sequence information, prolonged QT interval information, or blood analyte information.

In Example 20, the subject matter of any one of Examples 1-19 can include the communication circuit being configured to communicate directly or indirectly, to an external renal monitoring or therapy device, triggering information that is configured to adjust or direct the renal or monitoring therapy, including at least one of the following: ultrafiltration, infusion, or dialysis.

In Example 21, the subject matter of any one of Examples 1-20 can include logging (e.g., at the cardiac function management device) information received from our about the renal monitoring or therapy device.

Example 22 can include, or can be combined with any one of Examples 1-21 to include, subject matter that can include (such as an system, apparatus, method, tangible machine readable medium, etc.) detecting a tachyarrhythmia vulnerable period using at least one of an ambulatory cardiac function management device or an renal therapy device. In response to the detecting, triggering information can be communicated to the other of the cardiac function management device or the renal therapy device to trigger such other of the cardiac function management device or the renal therapy device to switch from a first operating mode to a second operating mode, wherein the second operating mode is configured for use during the tachyarrhythmia vulnerable period.

In Example 23, the subject matter of any one of Examples 1-22 can include switching the cardiac function management device from the first operating mode to the second operating mode. The second operating mode of the cardiac function management device can include adjusting, with respect to the first operating mode, at least one of pacing rate, electrostimulation energy, electrostimulation electrode configuration, interelectrode electrostimulation His-bundle pacing, or anti-tachyarrhythmia therapy.

In Example 24, the subject matter of any one of Examples 1-23 can include the renal monitoring or therapy device being configured to adjust an operating mode of the cardiac function management device using information about at least one of: (1) initiation of renal therapy; (2) adjustment of ongoing renal therapy; (3) cessation of renal therapy; or (4) a physiologic condition indicated by the renal monitoring or therapy device.

In Example 25, the subject matter of any one of Examples 1-24 can include logging (e.g., at the renal device) information received from or about the cardiac function management device.

In Example 26, the subject matter of any one of Examples 1-25 can include adjusting renal therapy in response to information received from or about the cardiac function management device.

The above detailed description includes references to the accompanying drawings, which form a part of the detailed description. The drawings show, by way of illustration, specific embodiments in which the invention can be practiced. These embodiments are also referred to herein as “examples,” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. An apparatus, comprising:
an ambulatory cardiac function management device comprising:
a data input configured to receive information about or from an external renal monitoring or therapy device;
a control circuit, comprising first and second operating modes, the first operating mode controlling operation of the implantable cardiac function management device in the absence of triggering information about or from the renal monitoring or therapy device, and the second operating mode controlling operation of the implantable cardiac function management device in response to receiving triggering information about or from the renal monitoring or therapy device; and
wherein the second operating mode includes an exit condition, returning control of operation of the implantable cardiac function management device to the first operating mode, when a vulnerable period, including a time period corresponding to an increased risk of tachyarrhythmia or intradialytic hypotension, has elapsed.

2. The apparatus of claim 1, wherein the control circuit is configured to adjust or direct therapy, provided by the cardiac function management device, in response to the triggering information received from the renal monitoring or therapy device.

3. The apparatus of claim 2, wherein the control circuit is configured to adjust or direct an anti-tachyarrhythmia therapy, provided by the cardiac function management device, in response to the triggering information received from the renal monitoring or therapy device.

4. The apparatus of claim 1, wherein the control circuit is configured to adjust or direct a physiological sensor of the cardiac function management device in response to the triggering information received from the renal monitoring or therapy device.

5. The apparatus of claim 4, wherein the control circuit is configured to qualify or flag data from the physiological sensor of the cardiac function management device using the triggering information received from the renal monitoring or therapy device.

6. The apparatus of claim 1, wherein the control circuit is configured to adjust determination of a thoracic fluid accumulation status or a congestive heart failure status in response to the triggering information received from the renal monitoring or therapy device.

7. The apparatus of claim 1, wherein the operating mode of the cardiac function management device is adjusted in response to the triggering information received from the renal monitoring or therapy device, wherein the triggering information indicates at least one of: (1) initiation of renal therapy; (2) adjustment of ongoing renal therapy; (3) cessation of renal therapy; or (4) a physiologic condition indicated by the renal monitoring or therapy device.

8. The apparatus of claim 1, wherein the second operating mode of the cardiac function management device includes adjusting, with respect to the first operating mode, at least one of pacing rate, electrostimulation energy, electrostimulation electrode configuration, interelectrode electrostimulation delay, His-bundle pacing, or anti-tachyarrhythmia therapy.

9. An apparatus, comprising:
a renal monitoring or therapy device comprising:
a tachyarrhythmia vulnerability detection circuit, configured to monitor a patient physiological or device operational renal parameter that varies during or in response to renal therapy, and to determine whether the renal parameter indicates a vulnerable period corresponding to an increased risk of a present or future tachyarrhythmia episode; and
a communication circuit, configured to communicate directly or indirectly to an ambulatory cardiac function management device to provide a tachyarrhythmia vulnerability mode trigger configured to alter operation of the cardiac function management device in response to the trigger.

10. The apparatus of claim 9, wherein renal monitoring or therapy device includes a control circuit that is configured to direct therapy provided by the renal monitoring or therapy device in response to information received from the cardiac function management device about at least one of a tachyarrhythmia vulnerability of the subject, a fluid accumulation status of the subject, or a congestive heart failure status of the subject.

11. The apparatus of claim 9, wherein the renal monitoring or therapy device is configured to adjust or direct an anti-tachyarrhythmia therapy, provided by the cardiac function management device.

12. The apparatus of claim 9, wherein the renal monitoring or therapy device is configured to adjust determination of a thoracic fluid accumulation status or a congestive heart failure status by the cardiac function management device.

13. The apparatus of claim 9, wherein the renal monitoring or therapy device is configured to adjust an operating mode of the cardiac function management device using information about at least one of: (1) initiation of renal therapy; (2) adjustment of ongoing renal therapy; (3) cessation of renal therapy; or (4) a physiologic condition indicated by the renal monitoring or therapy device.

14. The apparatus of claim 9, wherein the control circuit is configured to adjust or direct the renal monitoring therapy, in response to the information received from the cardiac function management device, by providing a control signal to modify at least one of the following: dialysis, blood flow rate, dialysate flow rate, ultrafiltration, dialysate composition, or infusion.

15. An apparatus, comprising:
an implantable cardiac function management device comprising:
a tachyarrhythmia detection or prediction circuit, configured to detect the presence of an existing or a predicted future tachyarrhythmia episode; and
a communication circuit, configured to communicate directly or indirectly to an external renal monitoring or therapy device triggering information that is configured to alter operation of the renal monitoring or therapy in response to the triggering information.

16. The apparatus of claim 15, wherein the communication circuit is configured to communicate directly or indirectly to an external renal monitoring or therapy device triggering information indicating a tachyarrhythmia vulnerability including at least one of: heart rate, heart rate variability, premature ventricular contraction information, long-short contraction sequence information, prolonged QT interval information, or blood analyte information.

17. The apparatus of claim 16, wherein the communication circuit is configured to communicate directly or indirectly, to an external renal monitoring or therapy device, triggering information that is configured to adjust or direct the renal monitoring or therapy, including at least one of the following: ultrafiltration, infusion, or dialysis.

18. A method comprising:
detecting a tachyarrhythmia vulnerable period using at least one of an ambulatory cardiac function management device or an renal therapy device; and
in response to the detecting, communicating triggering information to the other of the cardiac function management device or the renal therapy device to trigger such other of the cardiac function management device or the renal therapy device to switch from a first operating mode to a second operating mode, wherein the second operating mode is configured for use during the tachyarrhythmia vulnerable period.

19. The method of claim 18, comprising switching the cardiac function management device from the first operating mode to the second operating mode, and wherein the second operating mode of the cardiac function management device includes adjusting, with respect to the first operating mode, at least one of pacing rate, electrostimulation energy, electrostimulation electrode configuration, interelectrode electrostimulation delay, His-bundle pacing, or anti-tachyarrhythmia therapy.

20. The method of claim 19, wherein the renal monitoring or therapy device is configured to adjust an operating mode of the cardiac function management device using information about at least one of: (1) initiation of renal therapy; (2) adjustment of ongoing renal therapy; (3) cessation of renal therapy; or (4) a physiologic condition indicated by the renal monitoring or therapy device.
